(19) United States
(12) Patent Application Publication
(10) Pub. No.: US 2002/0143306 A1
(43) Pub. Date: Oct. 3, 2002

(54) Breathable stretch-thinned films having enhanced breathability

(76) Inventors: John David Tucker, Roswell, GA (US); Bryon P. Day, Canton, GA (US)

Correspondence Address: Melanie I. Rauch, Pauley Petersen Kinne & Fejer, Suite 365, 2800 W. Higgins Road, Hoffman Estates, IL, 60195, US

(21) Appl. No.: 09/788,177

(22) Filed: Feb. 16, 2001

(51) Int. Cl. A61F 13/15; A61F 13/20

(52) U.S. Cl. 604/369; 604/370; 604/371; 604/372

(57) ABSTRACT

A highly breathable, stretch-thinned film having enhanced breathability includes a matrix polymer, a particulate filler, and an incompatible polymer that is thermodynamically incompatible with the matrix polymer. The incompatible polymer can be added to the film in the form of laminate waste material.

### FIELD OF THE INVENTION

This invention is directed to film compositions in which laminate waste can be included, resulting in enhanced breathability of the film.

### BACKGROUND OF THE INVENTION

Highly breathable stretch-thinned (HBS) films are those that contain a polymer matrix, such as a Ziegler-Natta catalyzed linear low density polyethylene (LLDPE) and/or ultra low density polyethylene (ULDPE), and a particulate filler, such as a calcium carbonate filler, and are stretched to cause breathability to water vapor. The calcium carbonate filler provides void-initiating particles which enhance void formation during stretching of the films. HBS films are highly breathable, but there is potential for their breathability to be enhanced even more.

HBS films are often thermally or adhesively laminated to a polyolefin nonwoven web, to form a breathable laminate. In the laminating processes, the production of a certain degree of laminate waste is unavoidable. Laminate waste can potentially be put to use rather than being discarded in landfills.

There is a need or desire for HBS films having enhanced breathability, and for breathable film/nonwoven laminates containing the films.

There is yet a further need or desire for a constructive use for laminate waste.

### SUMMARY OF THE INVENTION

The present invention is directed to improved highly breathable stretch-thinned (HBS) films and laminates containing them. More particularly, the breathability of HBS films can be enhanced through the inclusion of laminate waste material. The laminate waste material includes at least one incompatible polymer which can be dispersed within the polymer matrix. During film stretching, the incompatible polymer enhances void formation around the inorganic filler particles dispersed within the matrix polymer. In other words, the incompatible polymer and the matrix polymer form a phase segregated structure in which droplets or domains of the incompatible polymer are dispersed within the matrix polymer. When making conventional stretch-thinned breathable films, elongation leads to the formation of voids at the interface between the matrix polymer and the particulate filler due to poor interfacial adhesion between the matrix polymer and the filler. By dispersing a quantity of an incompatible polymer within the same matrix polymer, the void formation can be enhanced or magnified upon elongation, perhaps due to poor interfacial adhesion between the matrix polymer and the incompatible polymer.

The breathability of HBS films can also be enhanced through the inclusion of incompatible polymers that are not necessarily added in the form of laminate waste material. As used herein, the term “incompatible polymer” refers to any polymer that is thermodynamically incompatible with a matrix polymer of the HBS film. For example, polypropylene can be used as an incompatible polymer in a filled linear low density polyethylene (LLDPE) matrix. Due to the thermodynamic incompatibility of polypropylene and polyethylene polymers, in a filled matrix of polypropylene and polyethylene the void formation during film stretching (and consequent moisture vapor breathability) can be enhanced.

With the foregoing in mind, it is a feature and advantage of the invention to provide HBS films having enhanced breathability and film/nonwoven laminates containing them. It is another feature and advantage of the invention to provide a constructive use for laminate waste material.

### BRIEF DESCRIPTION OF THE DRAWINGS

FIG. 1 is a cross-sectional view of a single-layer, breathable film of the invention;

FIG. 2 is a cross-sectional view of a three-layer, breathable film of the invention;

FIG. 3 is a cross-section view of a laminate including the breathable film of the invention; and

FIG. 4 is a schematic diagram of a process for making the breathable film of the invention.

### DEFINITIONS

Within the context of this specification, each term or phrase below will include the following meaning or meanings.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Film” refers to a thermoplastic film made using a film extrusion process, such as a cast film or blown film extrusion process. The term includes apertured films, slit films, and other porous films which constitute liquid transfer films, as well as films which do not transfer liquid.

“Layer” when used in the singular can have the dual meaning of a single element or a plurality of elements.

“Linear low density polyethylene (LLDPE)” refers to polymers of ethylene and higher alpha olefin comonomers such as C₃-C₁₂ comonomers, and combinations thereof, having a density of about 0.900 to 0.935 grams/cm³.

“Nonwoven” and “nonwoven web” refer to materials and webs of material which are formed without the aid of a textile weaving or knitting process.

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Ultra low density polyethylene (ULDPE)” refers to polymers of ethylene and higher alpha-olefin comonomers such as C₃-C₁₂ comonomers, and combinations thereof, having a density of about 0.860 to less than 0.900 grams/cm³.

These terms may be defined with additional language in the remaining portions of the specification.

### DETAILED DESCRIPTION OF THE PRESENTLY PREFERRED EMBODIMENTS

Referring to FIG. 1, the highly breathable stretch-thinned (HBS) film 10 in one embodiment of the invention can be a monolayer film including a particulate filler 20 within a matrix polymer 22. An incompatible polymer is combined with the matrix polymer. As used herein, the term “incompatible polymer” refers to any polymer that is thermodynamically incompatible with the matrix polymer, i.e., a polymer that will not homogeneously mix with the matrix polymer to form a single phase, and forms a separate phase within the matrix polymer. The term “matrix polymer” refers to a continuous phase polymer component, or a continuous phase blend of two or more compatible, miscible polymers.

The filler particles 20 define voids 24 within the matrix polymer 22, such that when the film 10 is stretched, the voids 24 form around the filler particles 20. Many of the voids 24 essentially connect with one another or are separated by thin polymer membranes, thereby forming tortuous paths throughout the film 10. The film 10 is rendered breathable by virtue of the tortuous paths through which air can travel. Breathability of the film is believed to be enhanced by a phase segregated structure formed by the incompatible polymer and the matrix polymer. Elongation of the film 10 leads to the formation of voids at the incompatible polymer/matrix polymer interface due to poor interfacial adhesion between the incompatible polymer and the matrix polymer.

In an LLDPE matrix, the presence of polypropylene as an incompatible polymer forms a phase segregated structure due to the thermodynamic incompatibility of polypropylene and polyethylene polymers. The incompatible polymer may enhance the void formation around the inorganic (e.g. calcium carbonate) filler particles in an LLDPE matrix during stretching of the film. The void formation may initiate at a lower stretching ratio, and the voids may form more extensively, due to the presence of the incompatible polymer.

Laminate waste material can be added to matrix polymer 22 of the HBS film 10 to supply the film with an incompatible polymer, such as polypropylene. The polypropylene may be contributed by a nonwoven (e.g. spunbond) web in a film/nonwoven laminate. Laminate waste was found to improve the breathability of HBS film 10 made from virgin material by up to 44% overall, as shown in the Example below. Furthermore, by using laminate waste as an HBS film additive, the need to dispose of the laminate waste using alternative means is eliminated.

The breathability of the HBS film 10 having enhanced breathability is expressed as water vapor transmission rate (WVTR) and is suitably in a range of about 1,000 to 100,000 grams/m²-24 hours, more suitably in a range of about 3,000 to 30,000 grams/m²-24 hours, most suitably in a range of about 10,000 to 20,000 grams/m²-24 hours. The WVTR of the film 10 can be determined using the Mocon WVTR test procedure described below.

The matrix polymer may constitute about 20% to 80% by weight of the HBS film 10. More suitably, the matrix polymer may constitute about 25% to 65% by weight of the HBS film, most suitably about 30% to 50%. The particulate filler 20 may constitute about 20% to 80% by weight of the HBS film 10. More suitably, the particulate filler may constitute about 35% to 70% by weight of the HBS film, most suitably about 40% to 60%. The incompatible polymer may constitute about 0.1% to 25% by weight of the HBS film 10. More suitably, the incompatible polymer may constitute about 1% to 20% by weight of the HBS film, most suitably about 2% to 10%. The amount of incompatible polymer should be less than the amount of the matrix polymer, such that the matrix polymer is the primary polymer component.

Examples of suitable matrix polymers include, but are not limited to, linear low density polyethylene (LLDPE), ultra low density polyethylene (ULDPE), and combinations thereof. Other suitable matrix polymer components include conventional (branched) low density polyethylene, high density polyethylene, other polyolefin homopolymers and copolymers, and combinations thereof. A particularly suitable matrix polymer includes about 50% to 85% by weight (of the matrix polymer) linear low density polyethylene formed using a Ziegler-Natta catalyst, and about 20% to 50% by weight ultra low density polyethylene formed using a single-site (metallocene or constrained geometry) catalyst.

The filler particles 20 may be inorganic filler particles. Suitable inorganic filler particles include, without limitation, calcium carbonate, clays, silica, alumina, barium sulfate, sodium carbonate, talc, magnesium sulfate, titanium dioxide, zeolites, aluminum sulfate, diatomaceous earth, magnesium sulfate, magnesium carbonate, barium carbonate, kaolin, mica, carbon, calcium oxide, magnesium oxide, aluminum hydroxide and combinations of these particles. The mean diameter for the filler particles 20 should range from about 0.1 to 10 microns, preferably about 0.5 to 7.0 microns, most preferably about 0.8 to 2.0 microns.

The incompatible polymer can include any suitable polymer that is thermodynamically incompatible with the matrix polymer. Where the primary matrix polymer is polyethylene or a combination thereof, suitable incompatible polymers include, without limitation, polypropylene, a propylene polymer, a propylene-ethylene copolymer, polystyrene, nylon, and polyester. “Polypropylene” refers to propylene homopolymers as well as copolymers containing up to about 10% by weight ethylene or a C₄-C₂₀ alpha-olefin comonomer.

The HBS film 10 should have a thickness which facilitates breathability to water vapor and which also permits structural integrity. After stretching, the film 10 should have a thickness of about 5-100 microns, suitably about 10-80 microns, most suitably about 15-60 microns. The film 10 can be prepared using cast or blown film extrusion, or another suitable film-forming technique.

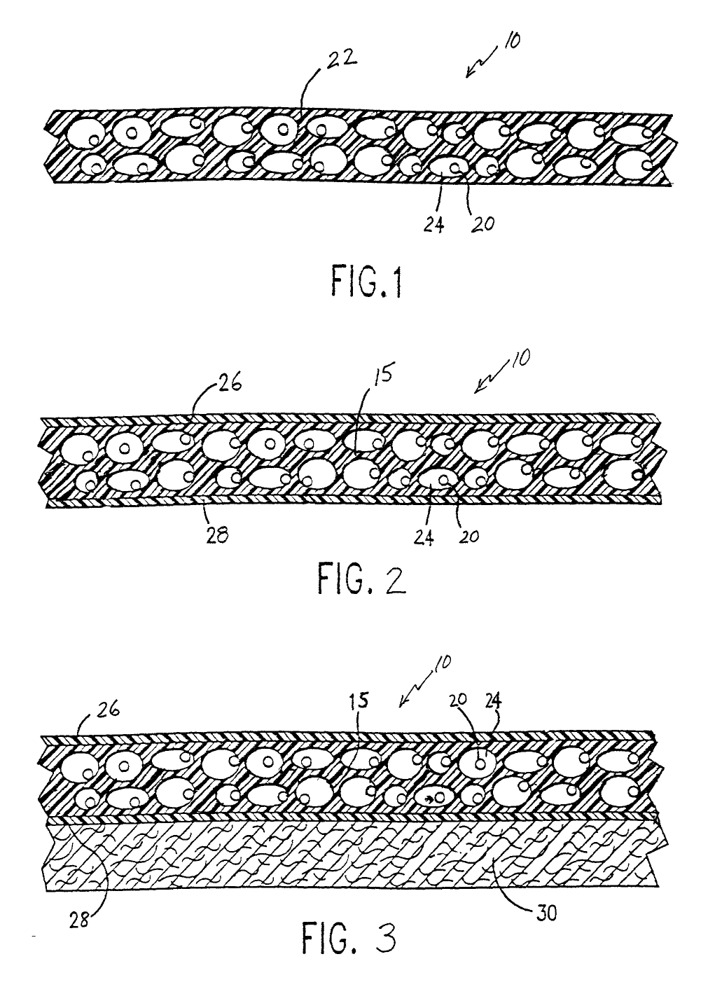

FIG. 2 illustrates another embodiment in which a multilayer breathable film 10 includes a primary breathable core layer 15 coextruded between two outer skin layers 26 and 28. In the multilayer film embodiments, the primary breathable core layer 15 should have essentially the same composition as the HBS film 10 described above. The core layer 15 can include the matrix polymer, the incompatible polymer, and the particulate filler 20. Either one or both of the skin layers 26, 28 can be a homopolymer, a blend of polymers, a polymer/filler or a blend/filler structure. The skin layers 26, 28 can be of the same composition as one another or of different compositions. For example, the first outer skin layer 26 can include only a thermoplastic polymer, and can be free of particulate filler 20, and the second outer skin layer 28 can include a matrix polymer and particulate filler 20 within the matrix polymer.

The multilayer film 10 in FIG. 2 illustrates that the outer skin layers 26 and 28 may or may not contain a filler. Furthermore, the outer skin layers 26 and 28 may or may not contain an incompatible polymer. The core layer 15 may have the same or a similar polymer composition to the monolayer film 10 described with respect to FIG. 1. The outer layers 26 and 28 may contain a softer, lower melting polymer or polymer blend which renders the outer layers more suitable as heat seal bonding layers for thermally bonding the film to a nonwoven web. When the outer layer (e.g., 26) is free of filler, one objective is to alleviate the build-up of filler at the extrusion die lip which may otherwise result from extrusion of a filled monolayer film. When the outer layer (e.g., 28) contains filler particles and voids, one objective is to provide a suitable bonding layer without adversely affecting the overall breathability of the film 10. Multi-layer films and skin layers are described in more detail in U.S. Pat. No. 6,075,179 issued to McCormack et al. and is hereby incorporated by reference.

The thickness and composition of the outer layers 26 and 28 should be selected so as not to substantially impair the moisture transmission through the breathable core layer 15. This way, the breathable core layer 15 may determine the breathability of the entire film, and the outer layers will not substantially reduce or block the breathability of the film. To this end, the skin layers 26 and 28 should be less than about 10 microns thick, suitably less than about 5 microns thick, desirably less than about 2.5 microns thick. Suitable skin layer polymers include without limitation ethylene vinyl acetates, propylene vinyl acetates, ethylene methyl acrylates, polystyrene polyamides, other vapor-permeable polymers, and blends of these with each other and with other polyolefins.

The breathable film may be laminated to one or more fibrous nonwoven substrates, such as a spunbond web, meltblown web, or airlaid web, using conventional adhesive bonding or thermal bonding techniques known in the art. The type of substrate and bonding will vary depending on the particular end use application. An example of a laminate is shown in FIG. 3, wherein a nonwoven web 30 is laminated to a multi-layer breathable film 10 of the invention. In the embodiment shown, the web 30, which can be a spunbonded web, is bonded to a skin layer 28 of the multilayer film 10, which layer may contain particulate filler particles. The primary filler-containing layer 15 faces away from the nonwoven web 30. The lamination of the film to the nonwoven web may be accomplished using conventional thermal bonding or adhesive bonding techniques. The fibrous nonwoven web may be made from any of the polymers listed above for the breathable film. In particular, the spunbond web may be made from a suitable polyolefin (e.g., polyethylene or polypropylene), or another thermoplastic polymer.

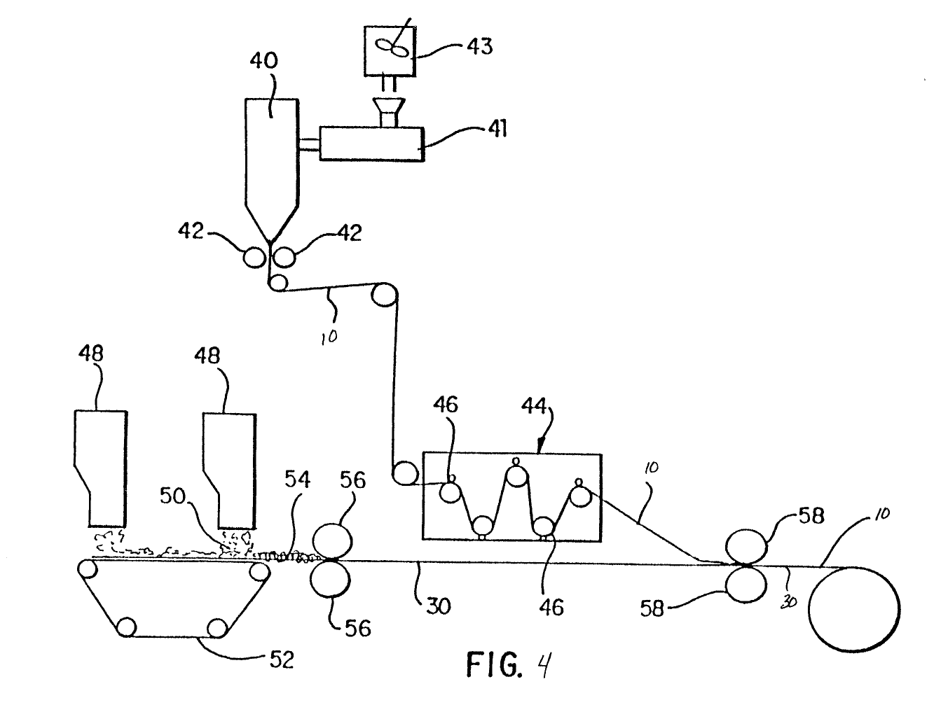

FIG. 4 illustrates an integrated process for forming an HBS film 10. Referring to FIG. 4, the film layer 10 is formed from a film extrusion apparatus 40 such as a cast or blown unit which could be in-line or off-line. Typically the apparatus 40 will include an extruder 41. Filled resin, including the matrix polymer, the particulate filler, and the incompatible polymer is prepared in a mixer 43 and directed to the extruder 41. The film layer 10 is extruded into a pair of nip or chill rollers 42, one of which may be patterned so as to impart an embossed pattern to the newly formed film layer 10.

From the film extrusion apparatus 40 or off-line rolls supplied, the filled film layer 10 is directed to a film stretching unit 44 which can be a machine direction orienter, commercially available from vendors including the Marshall and Williams Co. of Providence, R.I. The stretching unit 44 includes a plurality of pairs of stretching rollers 46, with each subsequent pair moving at a progressively faster speed than the preceding pair. The rollers 46 apply an amount of stress and progressively stretch the filled film layer 10 to a stretched length where the film becomes microporous and breathable. As shown, the film 10 can be stretched in the machine direction, which is the direction of travel of the film 10 through the process in FIG. 4.

Advantageously, the film 10 may be uniaxially stretched to about 1.1 to 7.0 times its original length, suitably to about 1.5 to 6.0 times its original length, more suitably to about 2.5 to 5.0 times its original length, using an elevated stretch temperature of about 150-200 degrees Fahrenheit for most polyolefin-based films. The elevated stretch temperature can be sustained by heating some of the stretch rollers 46. The optimum stretch temperature varies with the type of matrix polymer in the film 10, and is always below the melting temperature of the matrix polymer. The film 10 may also be biaxially stretched, using conventional techniques familiar to persons skilled in the art, with the cross-directional stretching occurring before, after or concurrently with the machine direction stretching.

Still referring to FIG. 4, the film layer 10 may be laminated to a nonwoven web 30 immediately after the film is stretched and immediately following manufacture of the nonwoven web. The nonwoven web 30 is suitably a polypropylene spunbond web and can be a spunbonded web, a meltblown web, a bonded carded web, or combination thereof. The web 30 can be formed by dispensing polymer filaments 50 from a pair of conventional spinnerettes 48, onto a conveyor assembly 52. The filaments 50 are deposited onto the conveyor to form a mat 54. The filaments 50 of the mat 54 are then compressed to form inter-filament bonding using a pair of nip rollers 56, resulting in a spunbonded web 30. The spunbonded web 30 is then transported to the calender bonding rollers 58 and is thermally bonded to one side of the film layer 10.

The HBS film 10 of the invention may be used in a wide variety of personal care absorbent articles and medical products. The term “personal care absorbent products” includes without limitation diapers, training pants, swim wear, absorbent underpants, baby wipes, adult incontinence products and feminine hygiene products. The term “medical products” includes without limitation medical garments, aprons, underpads, bandages, drapes and wipes.

A comparison of the breathability of a couple of HBS films in accordance with the invention is shown in the Example below.

### EXAMPLE

HBS films were prepared using the process described above with respect to FIG. 4. Each HBS film had the following virgin composition (excluding laminate waste), in percentages by weight: the core included 60.0% CaCO₃, available from Imerys of Sandersville, Ga. under the trade name FilmLink Supercoat; 20% LLDPE, available from Dow Chemical Co. of Midland, Mich., under the trade designation DOWLEX® 2517; and 20% LLDPE, available from Dow Chemical Co. under the trade designation DOWLEX® 2047AC; each of two skin layers included 50% polyolefin copolymer, available from Bassel Polymers of Wilmington, Del. under the trade designation Adflex KS-357P; and 50% poly (ethylene-co-vinyl acetate) from Exxon-Mobil Chemical Company of Baytown, Tex., under the trade designation LD-768.36. The skin layers made up 3% by volume of the ABA skin/core/skin laminate.

Laminate waste material (“reclaim”) was included as 10% of one of the samples in each trial, as indicated. The laminate waste material had the following composition, in percentages by weight: 37.20% CaCO₃, 35.80% polypropylene, 20.30% LLDPE, 3.36% ethylene vinyl acetate, and 3.36% copolymer. The level of CaCO₃ in each of the test samples was maintained at 60% by removing some of the CaCO₃ from the “virgin” composition. Each film sample was stretched to 4.26 times its original length in the machine direction, and each stretched film sample had a thickness of about 2 mils (50 microns). The film samples were tested for breathability using the Mocon Test Procedure for Water Vapor Transmission Rate (below). Data extracted from a set of test runs in which the films were stretched to 4.26 times their original length is shown in Table 1 below.

TABLE 1
Breathability of Modified HBS Film Stretched 4.26X

| | | Average | Std Dev | |
|---|---|---|---|---|
| | | (grams/m²-24 | (grams/m²-24 | Change in |
| Sample | Reclaim | hours) | hours) | WVTR |
| Control | 0% | 10094.32 | 647.95 | — |
| With Reclaim | 10% | 14584.67 | 1757.87 | +44% |

As can be seen in Table 1, the breathability of the HBS film sample containing 10% laminate waste material increased by 44%, compared to the similar HBS film without reclaim. Since the level of CaCO₃ in both the control sample and the test sample was held constant, the improved breathability in the test sample was, therefore, attributable to the presence of polypropylene.

A second set of data extracted from a second set of test runs in which the same types of films as those shown in Table 1 were stretched to 4.26 times their original length is shown in Table 2 below.

TABLE 2
Breathability of Modified HBS Film Stretched 4.26X

| | | Average | Std Dev | |
|---|---|---|---|---|
| | | (grams/m²-24 | (grams/m²-24 | Change in |
| Sample | Reclaim | hours) | hours) | WVTR |
| Control | 0% | 10974.87 | 833.89 | — |
| With Reclaim | 10% | 15250.38 | 1492.82 | +39% |

The results in Table 2 mirror the results in Table 1, with breathability of the sample increasing by 39% following the inclusion of 10% laminate waste.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

Mocon Test Procedure for Water Vapor Transmission Rate (WVTR)

A suitable technique for determining the WVTR (water vapor transmission rate) value of a film or laminate material of the invention is the test procedure standardized by INDA (Association of the Nonwoven Fabrics Industry), number IST-70.4-99, entitled “STANDARD TEST METHOD FOR WATER VAPOR TRANSMISSION RATE THROUGH NONWOVEN AND PLASTIC FILM USING A GUARD FILM AND VAPOR PRESSURE SENSOR” which is incorporated by reference herein. The INDA procedure provides for the determination of WVTR, the permeance of the film to water vapor and, for homogeneous materials, water vapor permeability coefficient.

The INDA test method is well known and will not be set forth in detail herein. However, the test procedure is summarized as follows. A dry chamber is separated from a wet chamber of known temperature and humidity by a permanent guard film and the sample material to be tested. The purpose of the guard film is to define a definite air gap and to quiet or still the air in the air gap while the air gap is characterized. The dry chamber, guard film, and the wet chamber make up a diffusion cell in which the test film is sealed. The sample holder is known as the Permatran-W Model 100K manufactured by Mocon/Modem Controls, Inc., Minneapolis, Minn. A first test is made of the WVTR of the guard film and the air gap between an evaporator assembly that generates 100% relative humidity. Water vapor diffuses through the air gap and the guard film and then mixes with a dry gas flow which is proportional to water vapor concentration. The electrical signal is routed to a computer for processing. The computer calculates the transmission rate of the air gap and the guard film and stores the value for further use.

The transmission rate of the guard film and air gap is stored in the computer as CalC. The sample material is then sealed in the test cell. Again, water vapor diffuses through the air gap to the guard film and the test material and then mixes with a dry gas flow that sweeps the test material. Also, again, this mixture is carried to the vapor sensor. The computer then calculates the transmission rate of the combination of the air gap, the guard film, and the test material. This information is then used to calculate the transmission rate at which moisture is transmitted through the test material according to the equation:

TR⁻¹_{test material}=TR⁻¹_{test material, guardfilm, airgap}−TR⁻¹_{guardfilm, airgap}

Calculations:

WVTR: The calculation of the WVTR uses the formula:

WVTR=Fpₛₐₜ(T)RH/Apₛₐₜ(T)(1−RH))

where:

F=The flow of water vapor in cc/min.,

pₛₐₜ(T)=The density of water in saturated air at temperature T,

RH=The relative humidity at specified locations in the cell,

A=The cross sectional area of the cell, and,

pₛₐₜ(T)=The saturation vapor pressure of water vapor at temperature T.

We claim:

1. A breathable, stretch-thinned film, comprising:
a matrix polymer;
a calcium carbonate filler; and
a polymer incompatible with the matrix polymer.

2. The stretch-thinned film of claim 1, wherein the matrix polymer comprises a linear low density polyethylene.

3. The stretch-thinned film of claim 1, wherein the matrix polymer comprises an ultra low density polyethylene.

4. The stretch-thinned film of claim 1, wherein the polymer incompatible with the matrix polymer comprises a propylene polymer.

5. The stretch-thinned film of claim 1, wherein the polymer incompatible with the matrix polymer comprises polypropylene.

6. The stretch-thinned film of claim 1, wherein the polymer incompatible with the matrix polymer comprises a propylene-ethylene copolymer.

7. The stretch-thinned film of claim 1, wherein the polymer incompatible with the matrix polymer comprises polystyrene.

8. The stretch-thinned film of claim 1, wherein the polymer incompatible with the matrix polymer comprises nylon.

9. The stretch-thinned film of claim 1, wherein the polymer incompatible with the matrix polymer comprises polyester.

10. The stretch-thinned film of claim 1, wherein the film comprises a laminate waste material which includes the polymer incompatible with the matrix polymer.

11. An absorbent article comprising the stretch-thinned film of claim 1.

12. A breathable, stretch-thinned film, comprising at least one layer, the layer including:
between about 20% and 80% a matrix polymer;
between about 20% and 80% a particulate inorganic filler; and
between about 0.1% and 25% a polymer incompatible with the matrix polymer.

13. The stretch-thinned film of claim 12, wherein the matrix polymer comprises a linear low density polyethylene.

14. The stretch-thinned film of claim 12, wherein the matrix polymer comprises an ultra low density polyethylene.

15. The stretch-thinned film of claim 12, wherein the matrix polymer comprises a linear low density polyethylene and an ultra low density polyethylene.

16. The stretch-thinned film of claim 12, wherein the polymer incompatible with the matrix polymer comprises a propylene polymer.

17. The stretch-thinned film of claim 12, wherein the polymer incompatible with the matrix polymer comprises polypropylene.

18. The stretch-thinned film of claim 12, wherein the polymer incompatible with the matrix polymer comprises a propylene-ethylene copolymer.

19. The stretch-thinned film of claim 12, wherein the polymer incompatible with the matrix polymer comprises polystyrene.

20. The stretch-thinned film of claim 12, wherein the polymer incompatible with the matrix polymer comprises nylon.

21. The stretch-thinned film of claim 12, wherein the polymer incompatible with the matrix polymer comprises polyester.

22. The stretch-thinned film of claim 12, wherein the film comprises a laminate waste material which includes the polymer incompatible with the matrix polymer.

23. The stretch-thinned film of claim 12, wherein the matrix polymer comprises between about 25% and 65% of the film layer.

24. The stretch-thinned film of claim 12, wherein the matrix polymer comprises between about 30% and 50% of the film layer.

25. The stretch-thinned film of claim 12, wherein the particulate inorganic filler comprises between about 35% and 70% of the film layer.

26. The stretch-thinned film of claim 12, wherein the particulate inorganic filler comprises between about 40% and 60% of the film layer.

27. The stretch-thinned film of claim 12, wherein the polymer incompatible with the matrix polymer comprises between about 1% and 20% of the film layer.

28. The stretch-thinned film of claim 12, wherein the polymer incompatible with the matrix polymer comprises between about 2% and 10% of the film layer.

29. The stretch-thinned film of claim 12, wherein the film further comprises at least one skin layer.

30. The stretch-thinned film of claim 12, wherein the film further comprises at least two skin layers.

31. An absorbent article comprising the stretch-thinned film of claim 12.

32. A breathable laminate, comprising:
a breathable film including a matrix polymer, a polymer incompatible with the matrix polymer, and a particulate filler; and
a nonwoven web.

33. The breathable laminate of claim 32, wherein the matrix polymer comprises a linear low density polyethylene.

34. The breathable laminate of claim 33, wherein the matrix polymer further comprises an ultra low density polyethylene.

35. The breathable laminate of claim 32, wherein the polymer incompatible with the matrix polymer comprises a propylene polymer.

36. The breathable laminate of claim 32, wherein the polymer incompatible with the matrix polymer comprises polypropylene.

37. The breathable laminate of claim 32, wherein the polymer incompatible with the matrix polymer comprises a propylene-ethylene copolymer.

38. The breathable laminate of claim 32, wherein the polymer incompatible with the matrix polymer comprises polystyrene.

39. The breathable laminate of claim 32, wherein the polymer incompatible with the matrix polymer comprises nylon.

40. The breathable laminate of claim 32, wherein the polymer incompatible with the matrix polymer comprises polyester.

41. The breathable laminate of claim 32, wherein the film comprises a laminate waste material which includes the polymer incompatible with the matrix polymer.

42. The breathable laminate of claim 32, wherein the nonwoven web comprises a spunbond web.

43. The breathable laminate of claim 32, wherein the nonwoven web comprises a meltblown web.

44. The breathable laminate of claim 32, wherein the nonwoven web comprises an air laid web.

45. An absorbent article comprising the breathable laminate of claim 32.
